(19) United States
(12) Patent Application Publication
(10) Pub. No.: US 2010/0145137 A1
(43) Pub. Date: Jun. 10, 2010

(54) Vaginal Device

(76) Inventor: Gaynor Morgan, Oostende (BE)

Correspondence Address: Tod T. Tumey, P.O. BOX 22188, HOUSTON, TX, 77227-2188, US

(21) Appl. No.: 12/279,195

(22) PCT Filed: Feb. 15, 2007
(86) PCT No.: PCT/GB2007/000517

§ 371 Date: Sep. 7, 2009

(30) Foreign Application Priority Data
Feb. 17, 2006 (GB) 0603186.3

(51) Int. Cl. A61F 2/50; A63B 23/20

(52) U.S. Cl. 600/29; 482/91

(57) ABSTRACT

The device for reducing the effects of stress incontinence and/or strengthening pelvic floor muscles has a body and a retraction element connected at an end of the body. The body has an undulating surface profile in the longitudinal direction which stimulates the outer and inner pelvic muscles to work to grip and massage the device. The device can include a recess for receiving the cervix.

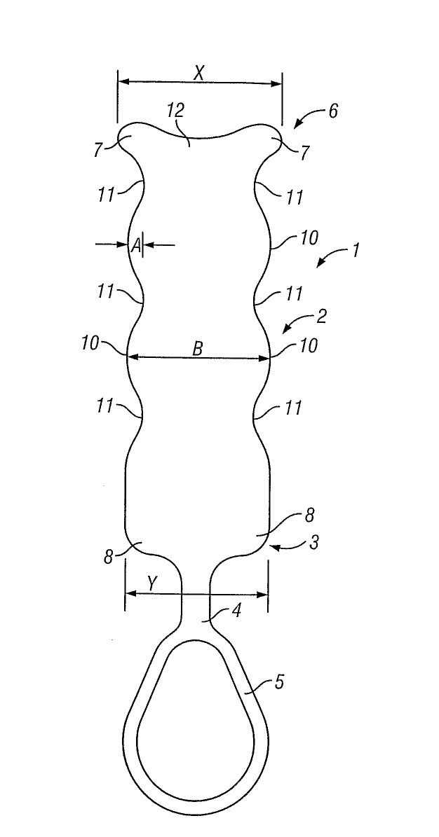

The present invention relates to a vaginal device for reducing the effect of stress incontinence and/or strengthening pelvic floor muscles, the device being for insertion in the vagina.

vaginal devices for use in reducing stress incontinence are disclosed in, for example, GB-A-2364645 and EP-A-1493400.

An improved device has now been devised.

According to a first aspect, the present invention provides a vaginal device for reducing the effect of stress incontinence and/or strengthening pelvic floor muscles, the device being for insertion in the vagina and comprising an elongate body portion and a retraction element provided at a proximal end of the body portion, the elongate body portion having:
i) an undulating surface profile in the longitudinal direction; and/or
ii) a first shoulder at the proximal end of the body and a second shoulder proximate the distal end of the body, the second shoulder being wider than the first shoulder; and/or
ii) the distal end of the body including a recess for receiving the cervix.

It is preferred that the undulating surface profile comprises a curve-form profile including concave and convex areas. Beneficially, the undulating surface profile comprises a series of surface crests and troughs alternating on the surface and spaced in the longitudinal direction of the device.

Desirably, at least two crests and troughs are provided alternating and spaced along the length of the device, preferably, the undulating surface profile is provided on the surface of the body about substantially the entire surface.

Preferably, the undulating surface profile is provided intermediate the first and second shoulders. Advantageously, the first and/or second shoulders comprise part of the undulating surface profile of the body.

It is preferred that the first and second shoulders are rounded in profile.

Preferably, the body has a substantially circular cross section perpendicular to its longitudinal axis, and:
I) the undulations of the, surface profile are provided about substantially the entire circumference of the body; and/or
ii) the first and/or second shoulders are provided around the entire circumference of the body.

Beneficially, the device comprises a one piece moulded device.

The surface of the body portion preferably comprises a non absorbent material.

The retraction element beneficially comprises an extension element extending from the proximal end of the body portion. The proximal (free) end of the extension element may be provided with a finger grab or pull formation (such as a ring or ‘T bar’) in order to aid gripping for retraction of the device from the vagina.

The extension element may be flexible or flexibly connected to the body portion.

The recess for supporting the cervix preferably comprises a convexly curved recess defining a dished zone in the distal end of the body portion. The recess serves to cradle and support the cervix, which is particularly beneficial in situations of prolapse.

The invention will now be further described, by way of example only, and with reference to the accompanying figure which is a schematic sectional view of a device in accordance with the invention.

Referring to the drawing, the device 1 is moulded of a biologically inert, non-absorbent, material (such as a silicone material) and comprises a relatively rigid body portion 2 and connected to a proximal end 3 of the body 2, an elongate flexible extension 4. The flexible extension 4 acts as a tether in order to aid retraction of the device 1 from the vagina, and may be provided with a termination in the form of, for example, a finger ring 5. As an alternative to the finger ring 5 a ‘T bar’ may be provided or even a termination without formation may be provided. A medical grade silicone is preferred such as that commercially available under the name Nu-Sil. A 40 Shore hardness has been found to be suitable.

The body portion 2 is generally circular in section perpendicular to the longitudinal axis of the device. The upper (distal) end 6 of the body portion is provided with a rounded circumferential shoulder 7, and the proximal end 3 of the body portion 2 is provided with a rounded circumferential shoulder 8. The width dimension X(diameter)of the upper shoulder 7 is greater than the width dimension Y (diameter) of the lower shoulder 8. This is an advantageous technical feature the nature and purpose of which will be explained.

Intermediate the proximal and distal ends of the body portion 2, the body portion 2 is provided with a surface profile undulating in the longitudinal direction of the device. The undulating surface profile comprises a series of curve-form longitudinally spaced convex crests 10 and waist portions defined by concave troughs 11. Typically between 2 and four crests and troughs are provided along the length of the body portion, and the depth of the profile (from crest to trough dimension A) is substantially in the range 5% to 25% (more preferably 5% to 15%) of the diameter of the body proportion at the crest (dimension B). The contoured surface profile of the body portion in the longitudinal direction of the device is an advantageous technical feature, as will be explained.

The distal end 6 of the body portion is provided with a concave dished recess 12 between opposed limits of upper shoulder 7. The recess 12 serves to locate and support the cervix, which is advantageous particularly in the situation of an individual having a prolapsed womb.

The device of the present invention enables the body portion, to act as a rigid ‘splint’ and aid the body to function naturally during urination. When the bladder is full, the compression of the body portion 2 against the vaginal wall acts as a splint supporting the urethra, and preventing involuntary urine loss by closing the urethra. The use of non-absorbent materials allows the natural body fluids to secret without inhibiting the function of the device. The device when inserted in the vagina also acts to strengthen the pelvic floor muscles.

The outer pelvic muscle acts to try to expel the device as a foreign body, whereas the inner pelvic muscles can be worked to grip and massage the device to retain the insert in position. The undulating outer surface profile of the device, and the greater width of the upper shoulder 7 with respect to the lower shoulder 8 are structural features of the device that enhance the ability of the inner pelvic muscles to grip and massage the device and therefore aid in retention of the device in position and also strengthen the pelvic floor muscles. The dimensions of the troughs and crests need to be in a range suitable to effect gripping and massage. The undulating profile extends substantially over the major part of the body portion between the proximal and distal ends.

In the embodiment shown the waisted portions defined by the troughs 11 extend around the body portion at a constant level. In an alternative embodiment, the waist portion troughs 11 and the crests 10 may be spiral or helical form extending intermediate the proximal and distal ends. As mentioned above, in order to have the desired effect between 2 and 4 crests and troughs will be provided. In usable situations it is believed that certainly 10 or less (more beneficially 6 or less) series of crests/troughs will be utilised. In this way the device will have between two and four bulbous portions along a respective longitudinal edge of the body portion; the respective bulbous portion being defined by the respective crest 10. Beneficially, the ‘wavelength’ of the troughs corresponds to the ‘wavelength’ of the crests 11, by which it is meant that the trough to trough spacing corresponds substantially to the crest to crest spacing.

Non circular geometries maybe employed for the body portion 2, but this is generally less preferred.

What is claimed:

1. A vaginal device for reducing the effect of stress incontinence and/or strengthening pelvic floor muscles, the device being for insertion in a vagina and comprising an elongate body portion and a retraction element provided at a proximal end of the body portion, the elongate body portion having:
i) an undulating surface profile in a longitudinal direction; and/or
ii) a first shoulder at the proximal end of the body and a second shoulder proximate a distal end of the body, the second shoulder being wider than the first shoulder; and/or
ii) the distal end of the body including a recess for receiving a cervix.

2. A device according to claim 1, wherein the undulating surface profile comprises a curve-form profile including concave and convex areas.

3. A device according to claim 1, wherein the undulating surface profile comprises a series of surface crests and troughs alternating on the surface and spaced in the longitudinal direction of the device.

4. A device according to claim 3, wherein at least two crests and troughs are provided alternating and spaced along a length of the device.

5. A device according to claim 1, wherein the undulating surface profile is provided on the surface of the body about substantially the entire surface.

6. A device according to claim 1, wherein the undulating surface profile is provided intermediate the first and second shoulders.

7. A device according to claim 1, wherein the first and/or second shoulders comprise part of the undulating surface profile of the body.

8. A device according to claim 1, wherein the first and second shoulders are rounded in profile.

9. A device according to claim 1, wherein the body has a substantially circular cross section perpendicular to its longitudinal axis, and:
Ii) the undulations of the surface profile are provided about substantially an entire circumference of the body; and/or
ii) the first and/or second shoulders are provided around the entire circumference of the body.

10. A device according to claim 1, wherein the device comprises a one piece moulded device.

11. A device according to claim 1, wherein the surface of the body portion comprises a non absorbent material.

12. A device according to claim 1, wherein the retraction element comprises an extension element extending from the proximal end of the body portion.

13. A device according to claim 12, wherein the extension element is flexible or flexibly connected to the body portion.

14. A device according to claim 1, wherein the recess for supporting the cervix comprises a convexly curved recess defining a dished zone in the distal end of the body portion.

15. A method of reducing the effect of stress incontinence and/or strengthening pelvic floor muscles, the method comprising providing the device according to claim 1, the device being for insertion in the vagina.
